(19) United States
(12) Patent Application Publication
(10) Pub. No.: US 2022/0241193 A1
(43) Pub. Date: Aug. 4, 2022

(54) PHARMACEUTICAL COMPOSITIONS IN THE FORM OF GEL CONTAINING XYLOGLUCAN AND ALCOHOLS FOR THE CONTROLLED RELEASE OF ACTIVE INGREDIENTS

(71) Applicant: ALFASIGMA S.P.A., Bologna (IT)

(72) Inventors: Giuseppe Claudio Viscomi, Bologna (IT); Paola MAFFEI, Bologna (IT); Cristiana Bruno, Bologna (IT); Mascia Federici, Bologna (IT); Clelia Dispenza, Palermo (IT); Maria Antonietta Sabatino, Palermo (IT)

(73) Assignee: ALFASIGMA S.P.A., Bologna (IT)

(21) Appl. No.: 17/596,509

(22) PCT Filed: Jun. 11, 2020
(86) PCT No.: PCT/EP2020/066181

§ 371 Date: Dec. 13, 2021

(30) Foreign Application Priority Data
Jun. 21, 2019 (IT) 102019000009273

(51) Int. Cl. A61K 9/06; A61K 47/36; A61K 47/10; A61K 45/06

(52) U.S. Cl. CPC A61K 9/06; A61K 47/36; A61K 9/0024; A61K 45/06; A61K 47/10

(57) ABSTRACT

The invention relates to gel compositions comprising pharmaceutical active ingredients. The composition comprises xyloglucan at a concentration between 0.1% and 10.0% by total weight of the composition, together with a primary alcohol at a concentration between 20.0 and 50.0% by total weight of the composition. The invention also describes a process for the preparation of the composition and its use in the treatment of pathologies wherein a controlled release of the active principle is useful.

### FIELD OF THE INVENTION

The present invention relates to gel compositions comprising pharmaceutical active ingredients.

The composition comprises xyloglucan at a concentration between 0.1% and 10.0% together with an alcohol at a concentration between 5.0 and 50.0% by total weight of the composition.

The invention also describes a process for the preparation of the composition and the use of said composition in the treatment of pathologies wherein a controlled release of the active principle is useful. The composition may comprise one or more pharmaceutical or nutraceutical active ingredients and it may be administered by parenteral, subcutaneous, vaginal, buccal, oral, topical or rectal route. The composition can be administered by the use of appropriate medical devices.

### STATE OF THE ART

WO 2009/028764 describes a gel for the release of proteins obtained from a complex of macromolecules with a thermo-reversible polysaccharide in the presence of a salt with salting-out properties. The resulting formulation is liquid at room temperature turning into a gel when injected, from the gel the protein is released.

WO 1999/059549 describes injectable hydrogels based on alginate esters containing a therapeutic protein and a metal ion.

US 2002/0019336 describes a composition containing a mucopolysaccharide, such as chondroitin sulfate or hyaluronate, the composition is useful for the release of proteins and the formation of the gel occurs as the pH value changes.

Miyazaki S. et al. in J Contr Rel 56, 75, 1998 describes the release of indomethacin from thermo-reversible gels obtained by enzyme-partially degraded xyloglucan at concentrations between 1% and 2% by weight in water or phosphate buffer at pH 7.2. Kant A. et al in Pharmacology online 2: 28, 2011 describes the in situ formation of gels in which the formation of the gel depends on changes in temperature, pH, presence of ions and ultraviolet rays, enabling the release of an active principle in a controlled way. The gel can be formed with synthetic or natural polymers and can be administered by oral, ocular, rectal, vaginal or injectable route. This document reports that when xyloglucan in aqueous solutions is partially degraded with β-galactosidase, the resulting product has the property to be a thermo-reversible gel, wherein the gel formation occurs by cooling down from high temperatures. This phenomenon does not occur with the native xyloglucan.

In situ gels can comprise mucoadhesive polymers for the release of active ingredients into the mucous membranes as described in EP 3173067.

EP 1898876 describes mucoadhesive compositions containing xyloglucan in aqueous solution at a concentration between 0.05% and 5.0% by weight, together with glycerol at concentrations between 10.0% and 70.0% by weight, wherein the mucoadhesive compositions described are in solution, ovules, gel, vaginal lavage and spray. This patent does not describe the formation of in situ gel.

JP 6490134 describes gel compositions containing an aqueous solution of xyloglucan and at least one saccharide and/or an alcohol. The described composition comprises xyloglucan at a concentration between 0.1% and 15.0% by weight, an alcohol selected in the group consisting of a di-, tri-, tetra-, penta and hexahydric alcohol in an amount between 10.0% and 50.0% by weight and a water-soluble polymer. The described compositions refer to gel compositions for cosmetic and food use, wherein the aim is to maintain the gel obtained during the storage thereof.

Todaro S. et al. Chem. in Eng. Trans. 2016; 49:289-294, describes the rheological properties of xyloglucan depolymerized with high energy irradiation. The fractions of depolymerized xyloglucan were characterized by FT-IR and by the formation of gel in presence of different concentration of ethanol.

Pasta S. et al., in “I materiali biocompatibili per 1a medicina/Biomaterials for Medicine” 2014 (2014-01-01), Universitas Studiorum, XP055676486, pages 369-370, describes gel formation when native or depolymerized xyloglucan are mixed with Transcutol® in a relative ratio 4:1 and he states that this gel could be useful in tissue engineering for human application.

In the pharmaceutical field there is an increased need to find new pharmaceutical compositions able of releasing active ingredients in a controlled way over time, such as to maintain the plasma concentration in an effective level resulting in a reduced number of administrations over time.

In view of the above, the object of the present invention is a controlled release pharmaceutical or nutritional composition in gel form, containing one or more active ingredients. The gel is formed directly at the contact between a natural polymer, xyloglucan, and an alcohol. The pharmaceutical gel composition comprising an active pharmaceutical ingredient can be obtained by mixing the components before use, in situ or for the preparation of a pharmaceutical product to be stored at room temperature or refrigerated.

### SUMMARY OF THE INVENTION

The present invention relates to a controlled release composition in form of gel comprising a pharmaceutical active ingredient.

This composition comprises xyloglucan, a primary alcohol and one or more active ingredients.

The invention discloses a controlled release composition in form of gel, comprising one or more pharmaceutical active ingredients, xyloglucan and a primary alcohol, wherein the xyloglucan is at a concentration between 0.1% and 10.0% by total weight of the composition. The composition comprises a primary alcohol selected from the group consisting of 2-(2-ethoxyethoxy) ethanol (Transcutol®), ethanol, propanol, butanol, cetyl alcohol, stearyl alcohol, and cetylstearyl alcohol.

In an object, the primary alcohol is 2-(2-ethoxyethoxy) ethanol (Transcutol®).

The composition of the invention can comprise one or more pharmaceutical active ingredients selected from the group comprising anti-inflammatory, anti-fungal, antibiotic, mimetic antibiotic, growth factors, disinfectants, anticancer agents, proteins, peptides, humectants, natural ingredients or their mixtures.

In one aspect, the composition comprises an amount of xyloglucan between 0.1% and 10.0%, 2-(2-ethoxyethoxy) ethanol between 5.0% to 50.0% by total weight of the composition, together with pharmaceutical active ingredients. The composition is useful for releasing in a controlled way active ingredient with a topical and/or systemic effect.

The invention also describes a process for obtaining a composition comprising the preparation of a purified xyloglucan in an aqueous solution at a concentration from 0.1% to 10.0% by weight, with a primary alcohol solution at a concentration of 5.0% to 50.0% by weight wherein the active principle can be included in both solutions depending on the solubility.

### DETAILED DESCRIPTION OF THE INVENTION

The present invention describes a controlled release composition in form of gel, wherein the gel comprises pharmaceutical active ingredients which are released in a controlled and effective way.

The composition of the present invention is obtained by mixing an aqueous solution of xyloglucan (XG) with an alcohol, for example a primary alcohol. The pharmaceutical gel composition including a pharmaceutical active ingredient, can be obtained by mixing the two components just before use, in situ or for the preparation of a pharmaceutical product to be stored at room temperature or refrigerated.

The active pharmaceutical ingredients comprised in the composition, can be included in the aqueous solution of xyloglucan or in the alcoholic solution according to their solubility, without any limitation. The active ingredients can also be stored as such or diluted in aqueous solutions with or without xyloglucan and included in the gel at the time of administration.

The gel composition is formed by mixing xyloglucan and a primary alcohol selected from: 2-(2-ethoxyethoxy) ethanol, ethanol, propanol, butanol, cetyl alcohol, stearyl alcohol, and cetylstearyl alcohol.

In a preferred aspect, gel compositions consisting of xyloglucan and 2-(2-ethoxyethoxy) ethanol comprising pharmaceutical active ingredients are described. Xyloglucan is a polysaccharide derived from tamarind seeds and is composed of a (1-4)-β-D-glucan chain which has (1-6)-α-D-xylose bonds partially replaced by (1-2)-β-D-galactoxylose. Xyloglucan forms thermo-reversible gels in water when partially degraded by β-galactosidase, and the resulting product, in diluted aqueous solutions, has the property of gelling in reversible thermal conditions. Gel formation is possible when galactose removal exceeds 35%. Furthermore, the transition temperature is inversely proportional to the polymer concentration and the galactose removal. Such behavior does not occur with native xyloglucan. Xyloglucan can be obtained by extraction from plants such as the pea plant, soybean, rice, bamboo or tamarind seeds. Xyloglucan derived from tamarind seeds has a molecular weight of approximately 5,000 to 1,000,000 Da. The polymer is preferably obtained by extraction with alkaline solutions and then further purified by extraction in boiling water, centrifugation and sterilizing filtration.

The xyloglucan included in the composition of the present invention is a native xyloglucan, not enzyme-degraded, purified according to the purification process described in EP 1898876, and it is included in a concentration between 0.1% and 10.0% by total weight of the composition.

The gel composition, obtained by mixing an aqueous solution of xyloglucan at a concentration between 0.1% and 10.0% by weight and a primary alcohol selected from 2-(2-ethoxyethoxy) ethanol, ethanol, propanol, butanol, cetyl alcohol, stearyl alcohol and cetylstearyl alcohol at concentration between 5.0 and 50.0% by total weight of the composition, releases the active ingredients in a controlled way.

According to the invention, the gel composition is obtained by mixing an aqueous solution of xyloglucan at a concentration between 0.1% and 10.0% by weight and a primary alcohol selected from 2-(2-ethoxyethoxy) ethanol, ethanol, propanol, butanol, cetyl alcohol, stearyl alcohol and cetylstearyl alcohol at concentration between 20.0 and 50.0% by total weight of the composition.

The gel composition obtained by mixing an aqueous solution of xyloglucan at a concentration between 0.1% and 10.0% by weight and 2-(2-ethoxyethoxy) ethanol at concentration between 5.0 and 50.0% by total weight of the composition releases the active ingredients in a controlled way.

The composition comprises xyloglucan at a concentration between 0.1% and 10.0% by total weight of the composition and 2-(2-ethoxyethoxy) ethanol at a concentration between 20.0% and 30.0% by total weight of the composition.

The composition comprises xyloglucan at a concentration between 1.0% and 5.0% and 2-(2-ethoxyethoxy) ethanol at a concentration between 20.0% and 50.0% by total weight of the composition.

The composition comprises xyloglucan at a concentration between 1.0% and 5.0% and 2-(2-ethoxyethoxy) ethanol at a concentration between 20.0% and 30.0% by total weight of the composition.

By varying the amount of xyloglucan and 2-(2-ethoxyethoxy) ethanol in the composition, it is possible to obtain gels in different amount, characterized by different consistency, and able to modulate the release of the active ingredients over time. The composition of the present invention enables to have a flexible matrix useful to provides controlled releases according to the active principle, the pathology to be treated and the delivery site.

It has been found that the addition of a primary alcohol, in particular 2-(2-ethoxyethoxy) ethanol, at a concentration between 5.0% and 50.0% by weight, to the aqueous solution of xyloglucan, at a concentration by weight between 0.1% and 10, 0%, forms a gel in less than 20 seconds, preferably from 1 to 10 seconds.

The composition comprises xyloglucan at a concentration between 0.1% and 10.0% by weight, preferably between 0.5% to 8.0% by weight, more preferably between 1.0% and 5.0% by total weight of the composition. The composition comprising xyloglucan at concentrations between 0.1% and 10.0% by weight with 2-(2-ethoxyethoxy) ethanol at concentrations between 5.0% and 50.0% by weight, is characterized by a rheological behavior different in comparison to those obtained by a composition comprising xyloglucan (XG) and secondary alcohols at the same concentrations.

The gel composition comprising xyloglucan at concentrations between 0.1% and 10.0% by weight and 2-(2-ethoxyethoxy) ethanol at concentrations between 20.0% and 50.0% by weight, forms a gel in a percentage by weight greater than 50.0% with respect to the weight of the composition. Furthermore, the amount of gel formed by the composition comprising xyloglucan at concentration between 0.1 and 10.0% and 2-(2-ethoxyethoxy) ethanol between 20.0% and 50.0% is greater than that obtained at the same concentrations from a composition comprising xyloglucan and a secondary alcohol such as for example propylene glycol.

Amounts of xyloglucan less than 0.1% form an amount of gel, is in an amount less than 10.0% with respect to the weight of the solution.

The composition comprising xyloglucan in concentrations between 2.0% and 5.0% by weight and 2-(2-ethoxyethoxy) ethanol in concentrations between 20.0% and 50.0% (w/w) forms a gel separating from the solution, which is in an amount greater than 60.0% of the weight of the solution.

According to the invention the composition can comprise 4% (w/w) xyloglucan and 50% (w/w) 2-(2-ethoxyethoxy) ethanol.

According to the invention the composition can comprise xyloglucan at a concentration between 2.0% (w/w) and 5.0% (w/w) and 2-(2-ethoxyethoxy) ethanol at a concentration between 20.0% (w/w) and 30.0% (w/w) by total weight of the composition.

Compositions comprising xyloglucan at concentrations between 1.0% and 5.0% by weight and a secondary alcohol such as propylene glycol at concentrations between 20.0% and 50.0% (w/w) form a gel separating from the solution, in an amount less than 50.0% of the weight of the solution and the gel formation requires a longer time than that with 2-(2-ethoxyethoxy) ethanol.

The gel composition obtained by mixing 2-(2-ethoxyethoxy) ethanol at a concentration of between 5.0% and 50.0% (w/w) with an aqueous solution of xyloglucan, at a concentration between 0.1% and 10.0% (w/w) is obtained by mixing thereof in less than 20 seconds, preferably less than 10 seconds.

The rheological parameters are useful for defining the characteristics of a liquid or a solid. Namely, the moduli G′ and G″, respectively known as the Storage Modulus and Loss Modulus, are frequency-dependent material functions. G′ is representative of solid behavior, G″ of liquid behavior. G′ is aligned with deformation, in accordance with a typically elastic behavior, G″ is in accordance with a typically liquid behavior.

The composition of the invention in form of gel, is characterized by a Storage Modulus (G′), greater than a Loss Modulus (G″).

The composition comprising xyloglucan at a concentration between 0.1% and 10.0% by weight and 2-(2-ethoxyethoxy) ethanol between 5.0% and 50.0% by weight, is characterized by values of G′ decreasing as frequency decreases. This feature is maintained also after 24 hours.

The composition of the present invention is characterized at time T0 by G′ values between 2000 Pa and 500 Pa with a frequency between 100 rad/sec and 0.1 rad/sec applied.

The composition of the present invention is characterized by G′ values after 24 hours (T24) between 3000 Pa and 1000 Pa, with a frequency between 100 rad/sec and 0.1 rad/sec applied.

The composition comprising a concentration of xyloglucan between 0.1% and 10.0% by weight and 2-(2-ethoxyethoxy) ethanol between 5.0% and 50.0% (w/w) is characterized at Time zero (T0) by values of G″ between 500 Pa and 100 Pa, when a stress with a frequency between 100 rad/sec and 0.1 rad/sec applied.

The composition comprising a concentration of xyloglucan between 0.1% and 10.0% (w/w) and 2-(2-ethoxyethoxy) ethanol between 5.0% and 50.0% (w/w) is characterized at T24 by values of G″ between 300 Pa and 100 Pa, a frequency between 100 rad/sec and 0.1 rad/sec applied.

The composition comprising xyloglucan at the concentrations above described and a secondary alcohol, such as for example propylene glycol, is characterized by values of G′ lower than G″ and both lower than those obtained in a composition comprising xyloglucan and 2-(2-ethoxyethoxy) ethanol at T0. The gel containing propylene glycol is formed in a longer time than that containing 2-(2-ethoxyethoxy) ethanol and the formed gel is characterized by weak form. This is confirmed by the values of G′ which are lower than those obtained with 2-(2-ethoxyethoxy) ethanol both at T0 and after 24 hours.

The composition comprising xyloglucan at a concentration between 0.1% and 10.0% by weight and 2-(2-ethoxyethoxy) ethanol at a concentration between 5.0% and 50.0% by weight is characterized at T0 by viscosity values greater than those obtained with a composition comprising xyloglucan and propylene glycol at the same concentrations. The composition comprising xyloglucan at a concentration between 0.1% and 10.0% by weight and 2-(2-ethoxyethoxy) ethanol at a concentration between 5.0% and 50.0% by weight, at time T0, is characterized by viscosity values ranging from 1×10¹⁰ to 2×10³ mPa when a shear stress with a shear rate from 0 to 95 sec⁻¹ is applied.

The composition comprising xyloglucan at a concentration from 0.1% to 10.0% by weight and 2-(2-ethoxyethoxy) ethanol at a concentration between 5.0% and 50.0% by weight after 24 hours is characterized by viscosity values between 3×10⁷ and 1×10³ mPa·s when a shear stress with a shear rate of 0 to 95 sec⁻¹ is applied.

A comparative composition comprising xyloglucan and a secondary alcohol, such as propylene glycol, at the same concentrations, at T0 shows viscosity values lower than those obtained in the compositions containing 2-(2-ethoxyethoxy) ethanol. The viscosity values of this composition even after 24 hours are lower than those of the composition containing 2-(2-ethoxyethoxy) ethanol, by at least one order of magnitude. The composition comprising xyloglucan at a concentration between 0.1% and 10.0% by weight and 2-(2-ethoxyethoxy) ethanol between 5.0% and 50.0% by weight, can include other alcohols or esters which can slow down the release of active principles. Such additives can be, for example, natural or synthetic polymers, soluble in water or water-insoluble waxes. These compounds can be in a weight ratio between 1:50 and 10:1 with respect to xyloglucan in the aqueous solution.

The gels are formed when the xyloglucan aqueous solution is mixed with 2-(2-ethoxyethoxy) ethanol. The two solutions can therefore be stored in suitable containers to be used as needed and prepared by mixing xyloglucan and 2-(2-ethoxyethoxy) ethanol to form the gel at the site of action. The two components can be delivered in the desired site by forming in this site the gel able of releasing the active ingredient by the use of a system, or a medical device having two channels or two chambers containing the two solutions. Depending on its solubility in water, the active substance can be included in the aqueous solution of xyloglucan or in the alcoholic solution of 2-(2-ethoxyethoxy) ethanol.

A process for the preparation of the composition according to the invention comprises the steps of:
preparing an aqueous solution of purified xyloglucan at a concentration from 0.1% to 10.0% by weight, adding the obtained xiloglucan solution to a solution of primary alcohol at a concentration from 5.0% to 50.0% by weight, wherein the active ingredient may be comprised in both solutions depending on its solubility.

The solutions are mixed at the time of use, before the use or during the phase of gel preparation to be stored.

The gel composition containing xyloglucan at a concentration between 0.1% and 10.0% by weight and 2-(2-ethoxyethoxy) ethanol between 5.0% and 50.0% by weight and pharmaceutical or nutritional ingredient, wherein these ingredients are immediately included in the gel favors their stability, avoiding possible problems of degradation. Gel compositions comprising xyloglucan between 0.1% and 10.0% by weight and 2-(2-ethoxyethoxy) ethanol between 5.0% and 50.0% by weight can comprise one or more pharmaceutical active ingredients selected in the group comprising anti-inflammatory, anti-fungal, antibiotics, mimetic antibiotics, growth factors, disinfectants, anticancer agents, proteins, peptides, humectants and natural ingredients or their mixture.

The gel composition is useful for releasing peptides, proteins and any other active ingredient that can degrade when stored if formulated with other excipients.

The administrations can take place by enteral (oral, sublingual, rectal), parenteral (subcutaneous, intradermal), transcutaneous route. The compositions can also be administered vaginally, nasally or through the oropharyngeal mucosa.

For example, this composition can be useful for administering drugs that easily reach the affected site for example by inflammation or infection, injury, dehydration. The compositions may also include, for example, recombinant proteins such as human recombinant interferon (rIFN), rIFN alpha, rIFN beta and rIFN gamma, G-CSF, wherein the release takes place in a controlled manner with a total recovery of the administered amount. The ability of the composition according to the invention to release an active principle in a controlled way has been demonstrated by an exemplary experiment using rIFN alb as active principle, as below described. The amount of active IFN released by the system was measured after 1, 3, 5, 24 and 48 hours. Measurements have shown that after 5 hours the total amount of IFN released was about 26%, after 24 hours the total amount of IFN released was about 60%, and after 48 hour the IFN is fully released. Such release occurring in 24 and 48 hours enables to always have an effective amount of plasma concentration of the active principle with fewer administrations.

The invention also provides kits for delivering the methods described herein. The kits include a dose of therapeutic agents in suitable packaging.

### EXAMPLES

### Example 1: Determination of Xyloglucan Gel Formation with 2-(2-ethoxyethoxy) Ethanol

Aqueous solutions of xyloglucan at concentrations of 0.4%, 1.0%, 3.2% and 4.0% by weight were prepared.

The xyloglucan solutions were prepared by mixing the necessary amount of xyloglucan in water and leaving under stirring until completely dissolved.

The xyloglucan solutions were mixed with 2-(2-ethoxyethoxy) ethanol at the concentrations shown in the table, in graduated tubes. In less than 10 seconds after the mixing the two liquids, the formation of the gel was observed. Further to a visual evaluation, the gel amount was determined after centrifugation of the solution and the percentage of gel in the solution was assessed.

The following table shows the relative quantities of the components: xyloglucan, water and 2-(2-ethoxyethoxy) ethanol present in the solution and the amount of gel formed after mixing. The percentage of formed gel is obtained with respect to the total amount of the solution.

TABLE 1

| % Xyloglucan | % Water | %2-(2-ethoxyethoxy) | % Gel |
|---|---|---|---|
| (w/w) | (w/w) | ethanol (w/w) | (w/w) |
| 0.38 | 94.62 | 5 | 1.05 |
| 0.3 | 74.7 | 25 | 28.7 |
| 0.2 | 49.8 | 50 | 33.3 |
| 0.1 | 24.9 | 75 | 20.15 |
| 0.02 | 4.98 | 95 | 6.8 |
| 0.95 | 94.05 | 5 | 15.2 |
| 0.75 | 74.25 | 25 | 54.4 |
| 0.5 | 49.5 | 50 | 54.45 |
| 0.25 | 24.75 | 75 | 30.55 |
| 0.05 | 4.95 | 95 | 13.25 |
| 3.04 | 91.96 | 5 | 16.6 |
| 2.4 | 72.6 | 25 | 88.15 |
| 1.6 | 48.4 | 50 | 50.55 |
| 0.8 | 24.2 | 75 | 23.3 |
| 0.16 | 4.84 | 95 | 8.35 |
| 3.8 | 91.2 | 5 | 40 |
| 3 | 72 | 25 | 64.9 |
| 2 | 48 | 50 | 44.1 |
| 1 | 24 | 75 | 21.55 |
| 0.2 | 4.8 | 95 | 5.2 |

### Example 2: Determination of Xyloglucan Gel Formation with Glycol Propylene (Comparative Example)

Aqueous solutions of xyloglucan were prepared at concentrations of 0.4%, 1.0%, 3.2% and 4.0% by weight. The solutions were mixed with propylene glycol at the concentrations shown in the table. The xyloglucan solutions were made by mixing the necessary amount of xyloglucan in water and mixing until complete dissolution.

The xyloglucan solutions were mixed with propylene glycol at the concentrations shown in the table, in graduated tubes. Further to a visual evaluation, the gel amount was determined after centrifugation of the solution and the percentage of gel in the solution was assessed.

The following table reports the relative amounts of the components: xyloglucan, water and propylene glycol in the solution and the quantity of gel formed after mixing. The percentage of the formed gel is obtained with respect to the total amount of the solution.

TABLE 2

| % Xyloglucan | % Water | % Propylene | % Gel |
|---|---|---|---|
| (w/w) | (w/w) | glycol (w/w) | (w/w) |
| 0.38 | 94.62 | 5 | 2 |
| 0.3 | 74.7 | 25 | 19.1 |
| 0.2 | 49.8 | 50 | 37.65 |
| 0.1 | 24.9 | 75 | 35.35 |
| 0.02 | 4.98 | 95 | 11.95 |
| 0.95 | 94.05 | 5 | 7.5 |
| 0.75 | 74.25 | 25 | 33.3 |
| 0.5 | 49.5 | 50 | 44.55 |
| 0.25 | 24.75 | 75 | 30.75 |
| 0.05 | 4.95 | 95 | 12.5 |
| 3.04 | 91.96 | 5 | 33.7 |
| 2.4 | 72.6 | 25 | 56.25 |
| 1.6 | 48.4 | 50 | 70.5 |
| 0.8 | 24.2 | 75 | 31.5 |
| 0.16 | 4.84 | 95 | 6.1 |
| 3.8 | 91.2 | 5 | 27.6 |
| 3 | 72 | 25 | 60.8 |
| 2 | 48 | 50 | 59.85 |
| 1 | 24 | 75 | 33.1 |
| 0.2 | 4.8 | 95 | 7.55 |

### Example 3: Determination of the Modules G′ and G″ of the Composition Xyloglucan-2-(2-ethoxyethoxy) Ethanol

A volume of 40 ml of the aqueous solution of xiloglucan at concentration of 3.2% (w/w) was mixed with 10 ml 2-(2-ethoxyethoxy) ethanol. The solutions were loaded onto two syringes and directly extruded onto the rheometer plate and analyzed. The measurements of G′ and G″ were obtained by an Antoon Paar MCR101 rheometer conditioned at a temperature of 37° C., using a 50 mm flat cone. The measurements of G′ and G″ were performed at T0 and T24.

Table 3 reports the values of the modules G′ and G″ as the applied stress increases.

TABLE 3

| | Storage | Loss | Storage | Loss |
|---|---|---|---|---|
| | Modulus | Modulus G″ | Modulus G′ | Modulus G″ |
| Frequency | G′ (T0) | (T0) | (T24) | (T24) |
| [rad/s] | [Pa] | [Pa] | [Pa] | [Pa] |
| 100 | 1150 | 365.04 | 1893.4 | 251.88 |
| 63.1 | 1075.5 | 320.81 | 1828.5 | 233.08 |
| 39.8 | 1007.4 | 280.35 | 1761.2 | 217.48 |
| 25.1 | 951.99 | 239.27 | 1695.8 | 206.21 |
| 15.8 | 896.29 | 204.54 | 1636 | 197.12 |
| 10 | 861.62 | 175.53 | 1577.6 | 191.35 |
| 6.31 | 821.93 | 154.2 | 1521.3 | 185.55 |
| 3.98 | 802.85 | 129.89 | 1469.4 | 184.7 |
| 2.51 | 781.69 | 120.13 | 1414.8 | 181.28 |
| 1.58 | 766.91 | 113.52 | 1364.4 | 177.65 |
| 1 | 764.91 | 104.86 | 1308.3 | 180.72 |
| 0.631 | 766.91 | 100.94 | 1258 | 180.29 |
| 0.398 | 768.6 | 107.59 | 1220.1 | 183.13 |
| 0.251 | 778.46 | 106.41 | 1168 | 182.77 |
| 0.158 | 815.9 | 109.97 | 1138 | 170.4 |
| 0.1 | 1104.5 | 120.44 | 1112.2 | 166.16 |

### Example 4: Determination of Storage Modulus (G′) and Loss Modulus (G″) of the Composition Containing Xyloglucan and Propylene Glycol (Comparative Example)

The Example is carried out as in Example 3 and the values of Storage Modulus (G′) and Loss Modulus (G″) of the composition obtained by mixing 40 ml of the aqueous solution 3.2% (w/w) xyloglucan with 10 ml of 2-(2-ethoxyethoxy) ethanol were determined.

Table 4 shows the values of the modules G′ and G″ measured at time T0 and after 24 hours (T24).

TABLE 4

| | Storage | Loss | Storage | Loss |
|---|---|---|---|---|
| | Modulus | Modulus G″ | Modulus G′ | Modulus G″ |
| Frequency | G′ (T0) | (T0) | (T24) | (T24) |
| [rad/s] | [Pa] | [Pa] | [Pa] | [Pa] |
| 100 | 142.03 | 175.29 | 837.43 | 170.62 |
| 63.1 | 96.606 | 141.05 | 799.77 | 154.67 |
| 39.8 | 62.894 | 109.66 | 760.56 | 139.13 |
| 25.1 | 39.255 | 82.484 | 723.26 | 125.54 |
| 15.8 | 23.506 | 60.17 | 687.88 | 114.27 |
| 10 | 13.355 | 42.736 | 658.45 | 102.08 |
| 6.31 | 7.1791 | 29.591 | 631.71 | 93.689 |
| 3.98 | 3.8704 | 19.83 | 602.17 | 89.072 |
| 2.51 | 1.9388 | 13.13 | 581.87 | 81.43 |
| 1.58 | 0.95303 | 8.5891 | 558.19 | 75.859 |
| 1 | 0.4636 | 5.5399 | 539.03 | 71.59 |
| 0.631 | 0.23673 | 3.5711 | 515.81 | 75.731 |
| 0.398 | 0.12583 | 2.2806 | 502.23 | 66.3 |
| 0.251 | 0.070546 | 1.4495 | 484.94 | 62.991 |
| 0.158 | 0.029126 | 0.93052 | 474.18 | 63.64 |
| 0.1 | 0.029699 | 0.59432 | 463.08 | 68.4 |

### Example 5: Determination of the Viscosity of Compositions Comprising Xyloglucan and 2-(2-ethoxyethoxy) Ethanol

The viscosity values were obtained by an Antoon Paar MCR101 rheometer, with a 50 mm flat cone geometry and the measurements were performed at 37° C. Table 5 reports the viscosity values of the composition obtained by mixing 40 ml of the 3.2% (w/w) xyloglucan aqueous solution with 10 ml 2-(2-ethoxyethoxy) ethanol at T0 and after 24 hours.

TABLE 5

| Shear | Viscosity | |
|---|---|---|
| rate | (mPa · s) | |
| (1/s) | T0 | T24 |
| 0.00958 | 1.50 × 10⁷ | 2.20 × 10⁷ |
| 0.0172 | 6.44 × 10⁶ | 1.30 × 10⁷ |
| 0.0296 | 3.58 × 10⁶ | 7.42 × 10⁶ |
| 0.0509 | 2.14 × 10⁶ | 4.21 × 10⁶ |
| 0.0875 | 1.28 × 10⁶ | 2.41 × 10⁶ |
| 0.15 | 7.83 × 10⁵ | 1.41 × 10⁶ |
| 0.258 | 4.82 × 10⁵ | 8.45 × 10⁵ |
| 0.444 | 2.98 × 10⁵ | 5.25 × 10⁵ |
| 0.764 | 1.77 × 10⁵ | 3.31 × 10⁵ |
| 1.31 | 1.15 × 10⁵ | 2.12 × 10⁵ |
| 2.26 | 69493 | 1.34 × 10⁵ |
| 3.88 | 44152 | 83993 |
| 6.69 | 27064 | 48581 |
| 11.5 | 16767 | 26180 |
| 19.8 | 10496 | 12586 |
| 34 | 6678.7 | 5623.9 |
| 56.2 | 4541.5 | 2649.2 |
| 94.3 | 2995.3 | 1350 |

### Example 6: Determination of the Viscosity of Compositions Comprising Xyloglucan and Propylene Glycol (Comparative Example)

The viscosity values were obtained by an Antoon Paar MCR101 rheometer, with a 50 mm flat cone geometry and the measurements were performed at 37° C.

Table 6 reports the viscosity values of the composition obtained by mixing 40 ml of the 3.2% (w/w) aqueous solution of xyloglucan with 10 ml of propylene glycol at time T0 and after 24 hours.

TABLE 6

| Shear | Viscosity | |
|---|---|---|
| rate | (mPa · s) | |
| (1/s) | T0 | T24 |
| 0.00991 | 1.12 × 10⁶ | 4.46 × 10⁶ |
| 0.0171 | 1.01 × 10⁶ | 3.39 × 10⁶ |
| 0.0295 | 8.67 × 10⁵ | 2.30 × 10⁶ |
| 0.0507 | 7.09 × 10⁵ | 1.55 × 10⁶ |
| 0.0871 | 4.52 × 10⁵ | 1.04 × 10⁶ |
| 0.152 | 2.79 × 10⁵ | 4.94 × 10⁵ |
| 0.258 | 1.56 × 10⁵ | 3.39 × 10⁵ |
| 0.444 | 87819 | 2.30 × 10⁵ |
| 0.763 | 55029 | 1.55 × 10⁵ |
| 1.31 | 35316 | 1.04 × 10⁵ |
| 2.26 | 21461 | 68641 |
| 3.88 | 13608 | 44952 |
| 6.67 | 8996 | 28991 |
| 11.5 | 6152 | 18854 |
| 19.7 | 4050.8 | 12152 |
| 33.9 | 2672.9 | 7873.8 |
| 55.7 | 2037.6 | 5419.7 |
| 93.8 | 1526.8 | 3768.2 |

### Example 7: Determination of the Release of Recombinant Interferon (rIFN α2b) from xyloglucan-2-(2-ethoxyethoxy) Ethanol Gel

A recombinant IFN α2b solution, containing 2.6×10⁸ IU/mg was prepared in a 4% by weight xyloglucan solution at pH 7; the unitary composition is reported in Table 7.

TABLE 7

| | Unitary composition | | Composition |
|---|---|---|---|
| Component | unitaria per 1 g | | % (w/w) |
| Na₂HPO₄ | 1.3 | mg | 0.13 |
| NaH₂PO₄ H₂O | 1.8 | mg | 0.18 |
| Glycine | 10 | mg | 10 |
| Tween 80 | 0.1 | mg | 0.01 |
| Xyloglucan | 40 | mg | 4 |
| H₂O | q.s. to 1 g | | |
| rIFNα2b (2.6 × 10⁸ IU/mg) | 0.096 | ml | 0.0096 |
| pH 7 | | | |

rIFN α2b in the gel has a concentration of 25,000,000 IU/ml, so that 20,000,000 IU are present in the extruded gel.

In a double chamber syringe, 2 ml of solution containing xyloglucan according to Table 7 were loaded in one channel and 0.5 ml of 2-(2-ethoxyethoxy) ethanol was loaded in the other one. Following the extrusion from the syringe, a gel is immediately formed, in less than 10 seconds.

To evaluate the release of IFN, the gel was placed in 2% (w/w) culture medium (MEM), thermostated at 37° C. and assessed by a biological test for cytopathic effect (CPE).

TABLE 8

| Time (hrs) | IU/ml | Total IU | Released IU/h |
|---|---|---|---|
| 0 | 3,906 | 78,113 | / |
| 1 | 64,471 | 1,293,327 | 1,293,327 |
| 3 | 226,480 | 4,597,970 | 1,532,657 |
| 5 | 291,977 | 6,134,405 | 1,226,881 |
| 24 | 672,360 | 14,034,027 | 584,751 |
| 48 | 1,097,957 | 23,218,341 | 483,715 |
| 72 | 755,755 | 17,472,241 | 242,670 |
| 96 | 899,449 | 21,101,890 | 219,811 |
| 168 | 842,355 | 20,859,456 | 124,163 |

In vitro release kinetics indicate that IFN is fully released between 24 and 48 hours, with 100% recovery.

1. A controlled release composition in the form of gel comprising a pharmaceutical active ingredient, xyloglucan and a primary alcohol, wherein the xyloglucan is at a concentration between 0.1% (w/w) and 10.0% (w/w), the primary alcohol is at a concentration between 20.0% (w/w) and 50.0% (w/w) and is selected from the group consisting of: 2-(2-ethoxyethoxy) ethanol, ethanol, propanol, butanol, cetyl alcohol, stearyl alcohol and cetylstearyl alcohol.

2. The composition according to claim 1 wherein the alcohol is 2-(2-ethoxyethoxy) ethanol (Transcutol®).

3. The composition according to claim 2, wherein 2-(2-ethoxyethoxy) ethanol is at a concentration between 20.0% (w/w) and 30.0% (w/w).

4. The composition according to claim 2, wherein the xyloglucan is at a concentration between 1.0% (w/w) and 5.0% (w/w) and 2-(2-ethoxyethoxy) ethanol is at a concentration between 20.0% (w/w) and 50.0% (w/w).

5. The composition according to claim 4 wherein the xyloglucan is at a concentration between 1.0% (w/w) and 5.0% (w/w) and 2-(2-ethoxyethoxy) ethanol is at a concentration between 20.0% (w/w) and 30.0% (w/w).

6. The composition according to claim 2 wherein the xyloglucan is at a concentration between 2.0% (w/w) and 5.0% (w/w) and 2-(2-ethoxyethoxy) ethanol is at a concentration between 20.0% (w/w) and 50.0% (w/w).

7. The composition according to claim 6 wherein the xyloglucan is at a concentration between 2.0% (w/w) and 5.0% (w/w) and 2-(2-ethoxyethoxy) ethanol is at a concentration between 20.0% (w/w) and 30.0% (w/w).

8. The composition according to claim 1 comprising 4% (w/w) xyloglucan and 50.0% (w/w) 2-(2-ethoxyethoxy) ethanol.

9. The composition according to claim 1 characterized by a Storage Modulus (G′) value between 2000 Pa and 500 Pa at T0, and by a value between 3000 Pa and 1000 Pa after 24 hours, when subjected to a frequency between 100 rad/sec and 0.1 rad/sec.

10. The composition according to claim 1, characterized by a viscosity value between 1×10¹⁰ mPa·s and 2×10³ mPa·s at T0 and by a viscosity value between 3×10⁷ mPa·s and 1×10³ mPa·s at T24, when subjected to a shear rate between 0 sec⁻¹ and 95 sec⁻¹.

11. The composition according to claim 1 wherein the pharmaceutical active ingredient is selected from the group consisting of anti-inflammatory, antifungal, antibiotics, mimetic antibiotics, grow factors, disinfectants, anti-tumorals, proteins, peptides and humectants.

12. The composition according to claim 1 for administration by enteral, parenteral, transcutaneous or transmucosal route.

13. The composition according to claim 12 wherein the administration by enteral route is oral, sublingual and rectal administration, the administration by parenteral route is subcutaneous or intradermic administration, the administration by transcutaneous or transmucosal route is through vaginal, nasal or oropharyngeal mucosa.

14. A process for the preparation of the composition according to claim 1 comprising the steps of:
a) preparing an aqueous solution of purified xyloglucan at a concentration between 0.1% (w/w) and 10.0% (w/w);
b) adding the xyloglucan solution of step a) to a solution of primary alcohol at a concentration between 20.0% (w/w) and 50.0% (w/w), wherein the active ingredient may be comprised in both solutions depending on its solubility.

15. The process according to claim 14 wherein the solutions are mixed at the time of use, before the use or during the phase of gel preparation to be stored.

16. (canceled)

17. The composition according to claim 1 comprised in a medical device.
